(19) United States
(12) Patent Application Publication
(10) Pub. No.: US 2023/0343449 A1
(43) Pub. Date: Oct. 26, 2023

(54) SYSTEMS AND METHODS FOR EXTRACTION AND PROCESSING OF INFORMATION FROM IMAGING SYSTEMS IN A MULTI-VENDOR SETTING

(71) Applicant: KONINKLIJKE PHILIPS N.V., EINDHOVEN (NL)

(72) Inventors: Thomas Erik AMTHOR, HAMBURG (DE); Olga STAROBINETS, NEWTON, MA (US); Sandeep Madhukar DALAL, WINCHESTER, MA (US); Hareesh CHAMARTHI, CAMBRIDGE, MA (US); Tanja NORDHOFF, HAMBURG (DE)

(21) Appl. No.: 17/923,063

(22) PCT Filed: Apr. 27, 2021
(86) PCT No.: PCT/EP2021/060897

§ 371 Date: Nov. 3, 2022

Related U.S. Application Data
(60) Provisional application No. 63/023,276, filed on May 12, 2020.

(51) Int. Cl. G16H 40/67; G16H 80/00; G16H 30/20; G06V 20/50; G06T 11/60; G06V 30/18; G06V 10/56; G06V 20/40

(52) U.S. Cl. CPC G16H 40/67; G16H 80/00; G16H 30/20; G06V 20/50; G06T 11/60; G06V 30/18; G06V 10/56; G06V 20/46; G06V 20/48; G16H 40/20

(57) ABSTRACT

A non-transitory computer readable medium (26) stores instructions executable by at least one electronic processor (20) to perform a method (100) of providing assistance from a remote expert (RE) to a local operator (LO) of a medical imaging device (2) during a medical imaging examination. The method includes: extracting image features from image frames displayed on a display device (24′) of a controller (10) of the medical imaging device operable by the local operator during the medical imaging examination; converting the extracted image features into a representation (43) of a current status of the medical imaging examination; and providing a user interface (UI) (28) displaying the representation on a workstation (12) operable by the remote expert.

The following relates generally to the imaging arts, remote imaging assistance arts, remote imaging examination monitoring arts, and related arts.

### BACKGROUND

The increasing problem of getting highly qualified staff for performing complex medical imaging examinations has driven the concept of bundling medical expertise in remote service centers. The basic idea is to provide virtual availability of Senior Technologists as on-call expert in case a technologist or operator performing a medical imaging examination needs assistance with a scheduled examination or runs into unexpected difficulties. In either case, the remote expert would remotely assist the on-site operator by receiving real-time views of the situation by way of screen mirroring and one or more video feeds of the imaging bay. The remote expert typically would not directly operate the medical imaging device, but would provide advice or other input for assisting the local technologist.

To make such a remote service center commercially viable, it would be advantageous to enable the remote expert to concurrently assist (or be on call to assist) a number of different local technologists performing possibly concurrent medical imaging examinations. Preferably, the remote service center would be able to connect the expert to imaging systems of different models and/or manufactured by different vendors, since many hospitals maintain a heterogeneous fleet of imaging systems. This can be achieved by screen sharing or screen mirroring technologies that provide the remote expert a real-time copy of the imaging device controller display, along with video cameras to provide views of the imaging bay and, optionally, the interior of the bore or other examination region of the imaging device.

The remote expert is assumed to have experience and expertise with the different user interfaces of the different medical imaging systems and vendors for which the expert is qualified to provide assistance. When providing (potentially simultaneous) assistance to multiple imaging bays, the expert is expected to rapidly switch between the screen views of the different imaging systems to extract the required pieces of information for quickly assessing the situation in each imaging bay. This is challenging as required pieces of information may be differently located on differently designed user interfaces.

The following discloses certain improvements to overcome these problems and others.

### SUMMARY

In one aspect, a non-transitory computer readable medium stores instructions executable by at least one electronic processor to perform a method of providing assistance from a remote expert to a local operator of a medical imaging device during a medical imaging examination. The method includes: extracting image features from image frames displayed on a display device of a controller of the medical imaging device operable by the local operator during the medical imaging examination; converting the extracted image features into a representation of a current status of the medical imaging examination; and providing a user interface (UI) displaying the representation on a workstation operable by the remote expert.

In another aspect, an apparatus for providing assistance from a remote expert to a local operator during a medical imaging examination performed using a medical imaging device includes a workstation operable by the remote expert. At least one electronic processor is programmed to: extract image features from image frames displayed on a display device of a controller of the medical imaging device operable by the local operator during the medical imaging examination; convert the extracted image features into a representation of a current status of the medical imaging examination by inputting the image features into an imaging examination workflow model indicative of a current state of the medical imaging examination; and provide a UI displaying at least one of the representation and the imaging examination workflow model on the workstation operable by the remote expert.

In another aspect, a method of providing assistance from a remote expert to a local operator during a medical imaging examination includes: extracting image features from image frames displayed on a display device of a controller operable by the local operator during the medical imaging examination; converting the extracted image features into a representation indicative of a current status of the medical imaging examination by: identifying one of more of the extracted features from the image frames as personally identifiable information of a patient to be scanned during the medical imaging examination; and generating modified image frames from the image frames displayed on the display device of the controller by one of removing the identified personally identifiable information features from the image frames or replacing the personally identifiable information in the image frames with text, a symbol, or a color; inputting the representation into an imaging examination workflow model indicative of a current state of the medical imaging examination; and providing a UI displaying the modified image frames as a video feed, the abstract representation, and the imaging examination workflow model on a workstation operable by the remote expert.

One advantage resides in providing a remote expert or radiologist assisting a technician in conducting a medical imaging examination with situational awareness of local imaging examination(s) which facilitates providing effective assistance to one or more local operators at different facilities.

Another advantage resides in providing a remote expert or radiologist assisting one or more technicians in conducting a medical imaging examination with a list or other summary of relevant extracted information from shared screens of different medical imaging systems operated by technicians being assisted by the remote expert or radiologist.

Another advantage resides in providing a consistent user interface for the remote expert or radiologist of the shared screens operated by the technicians.

Another advantage resides in removing or blocking information related to a patient being imaged by a technician in data transmitted to a remote expert or radiologist.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 diagrammatically shows modules implemented by the apparatus of FIG. 1.

FIG. 3 shows an example of an output generated by the apparatus of FIG. 1.

FIG. 4 shows an example flow chart of operations suitably performed by the apparatus of FIG. 1.

### DETAILED DESCRIPTION

The following relates to Radiology Operations Command Center (ROCC) systems and methods, which provides remote “supertech” assistance to a local technician performing an imaging examination, and more particularly to a center that provides assistance to clients with imaging devices from multiple vendors. In this case, tracking the statuses of different imaging devices assigned to a given supertech can be difficult, since the statuses are presented using different device controller user interface (UI) formats, with the information arranged differently on the screen and amongst different UI tabs, and with quantitative information sometimes being presented in different units by imaging devices of different vendors. Furthermore, all information is not constantly displayed—for example, the user may go to a setup tab of the UI to input information about the patient and imaged anatomy, a scans tab to set up the scan list, and a current scan tab to set up and execute the current scan.

In some embodiments disclosed herein, a system provides screen capture, and uses vendor- and modality-specific templates along with optical character recognition (OCR) to identify and extract information from the displayed tabs of the UI as they are brought up. The extracted information is stored in a vendor-agnostic representation using a common (vendor-agnostic) set of units. The extracted information is also input to an imaging examination workflow model of the imaging process (for example, a state machine or a BPMN model) which tracks the current state of the imaging examination. The extracted information may also include any extracted warnings, alerts, or the like. The output of the vendor agnostic representation and the imaging examination workflow model for each imaging bay assigned to the supertech is displayed as a list that provides the supertech with a concise assessment of the state of each imaging bay at any given time, in a vendor-agnostic format.

While this list is useful, for providing assistance to a particular imaging bay the supertech needs to see the detailed controller display. However, in some contemplated commercial settings, the supertech should not see all information shown on the controller display. For example, patient-identifying information (PII) may be anonymized, and any windows showing non-imaging control related content (e.g., a window showing the display of another program running on the controller) may be blocked out. To implement this, the vendor- and modality-specific templates and OCR processing identify regions of the screen showing PII or other information that needs to be modified, and the captured screen frames are modified appropriately before presenting to the supertech.

In various embodiments disclosed herein, the image processing may be implemented at the client side and/or at the ROCC side. Client-side implementation may be preferable from the standpoint of ensuring removal of PII prior to the data stream being sent off-site; whereas, ROCC-side implementation may be more useful from a software updating standpoint. A mixed approach is also contemplated, e.g. PII removal might be performed client-side and the remaining processing implemented ROCC-side.

It should be noted that the ROCC is not necessarily centralized at a single geographical location. In some embodiments, for example, the ROCC may comprise remote experts drawn from across an entire state, country, continent, or even drawn from across the world, and the ROCC is implemented as a distributed Internet-based infrastructure that provides data transfer (e.g. screen sharing and video feed transfer) and telephonic and/or video communication connectivity between the various experts and the imaging bays being assisted by those experts, and tracks time of the provided assistance, outcomes, and/or other metrics for billing or auditing purposes as may be called for in a given commercial implementation. Furthermore, in addition to the ROCC application, the disclosed systems and methods could find use in providing a central monitoring station for a larger medical institution or network. In such settings, the disclosed approach could be used to provide a radiology manager an overview of all imaging bays. In this application, PII removal might (or might not) be unnecessary.

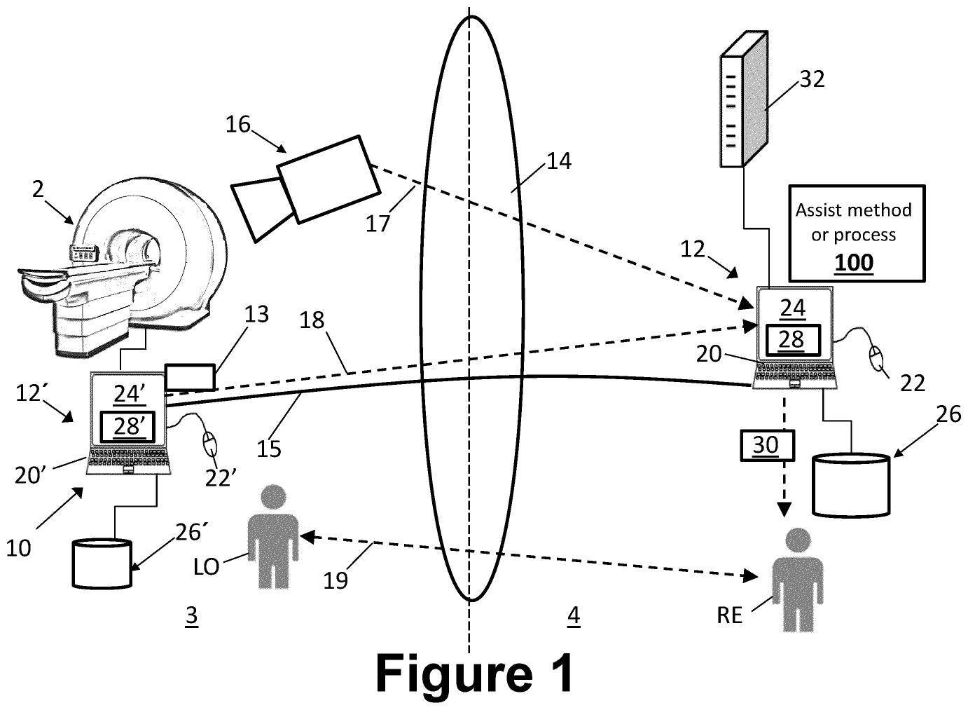

With reference to FIG. 1, an apparatus for providing assistance from a remote medical imaging expert RE (or supertech) to a local technician operator LO is shown. As shown in FIG. 1, the local operator LO, who operates a medical imaging device (also referred to as an image acquisition device, imaging device, and so forth) 2, is located in a medical imaging device bay 3, and the remote operator RE is disposed in a remote service location or center 4. It should be noted that the “remote operator” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote operator RE cannot directly visually observe the imaging device 2 in the imaging device bay 3 (hence optionally providing a video feed or screen-sharing process as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals, and a single remote expert RE may concurrently monitor and provide assistance (when required) for multiple imaging bays being operated by multiple local operators, only one of which local operator is shown by way of representative illustration in FIG. 1. The local operator controls the medical imaging device 2 via an imaging device controller 10. The remote operator is stationed at a remote workstation 12 (or, more generally, an electronic controller 12).

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote workstation 12) is in communication with multiple medical bays via a communication link 14, which typically comprises the Internet augmented by local area networks at the remote operator RE and local operator LO ends for electronic data communications.

As diagrammatically shown in FIG. 1, in some embodiments, a camera 16 (e.g., a video camera) is arranged to acquire a video stream 17 of a portion of the medical imaging device bay 3 that includes at least the area of the imaging device 2 where the local operator LO interacts with the patient, and optionally may further include the imaging device controller 10. The video stream 17 is sent to the remote workstation 12 via the communication link 14, e.g. as a streaming video feed received via a secure Internet link.

In other embodiments, the live video feed 17 is, in the illustrative embodiment, provided by a video cable splitter 15 (e.g., a DVI splitter, a HDMI splitter, and so forth). In other embodiments, the live video feed 17 may be provided by a video cable connecting an auxiliary video output (e.g. aux vid out) port of the imaging device controller 10 to the remote workstation 12 of the operated by the remote expert RE.

Additionally or alternatively, a screen mirroring data stream 18 is generated by a screen sharing or capture device 13, and is sent from the imaging device controller 10 to the remote workstation 12. The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone.

FIG. 1 also shows, in the remote service center 4 including the remote workstation 12, such as an electronic processing device, a workstation computer, or more generally a computer, which is operatively connected to receive and present the video 17 of the medical imaging device bay 3 from the camera 16 and to present the screen mirroring data stream 18 as a mirrored screen from the screen capture device 13. Additionally or alternatively, the remote workstation 12 can be embodied as a server computer or a plurality of server computers, e.g. interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices, e.g. one display presenting the video 17 and the other display presenting the shared screen of the imaging device controller 10 generated from the screen mirroring data stream 18. Alternatively, the video and the shared screen may be presented on a single display in respective windows. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24.

The medical imaging device controller 10 in the medical imaging device bay 3 also includes similar components as the remote workstation 12 disposed in the remote service center 4. Except as otherwise indicated herein, features of the medical imaging device controller 10, which includes a local workstation 12′, disposed in the medical imaging device bay 3 similar to those of the remote workstation 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the screen mirroring data stream 18 carries the content presented on the display device 24′ of the medical imaging device controller 10. The communication link 14 allows for screen sharing between the display device 24 in the remote service center 4 and the display device 24′ in the medical imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 or the mirroring data stream 18 and displayed on the remote workstation display 24 at the remote location 4.

FIG. 1 shows an illustrative local operator LO, and an illustrative remote expert RE (i.e. expert, e.g. supertech). However, in a Radiology Operations Command Center (ROCC) as contemplated herein, the ROCC provides a staff of supertechs who are available to assist a local operators LO at different hospitals, radiology labs, or the like. The ROCC may be housed in a single physical location, or may be geographically distributed. For example, in one contemplated implementation, the remote operators RO are recruited from across the United States and/or internationally in order to provide a staff of supertechs with a wide range of expertise in various imaging modalities and in various imaging procedures targeting various imaged anatomies. In other words, the ROCC may be located in the remote service center 4, with multiple remote workstations 12 operated by a corresponding number of remote experts RE. Furthermore, any given remote expert RE may be concurrently monitoring/assisting multiple imaging bays, possibly containing imaging devices of different makes (i.e., manufactured by different vendors) and/or models. In this working environment, it is important that the remote expert RE be able to quickly assess the status of any particular imaging bay assigned to the remote expert, and quickly determine any appropriate assistance that the remote expert RE may be able to provide to a particular assigned imaging bay. Conventionally, such multitasking is made more difficult by the differences in user interfaces of imaging devices of different makes/models. For example, relevant information may be presented on different screens of the user interfaces of different make/model imaging devices. Conventionally, such multitasking is also made more difficult by the fact that, due to the large amount of information handled via the imaging device controller, all information is not displayed at the same time. As a consequence, the mirror of the imaging device controller display at the workstation 12 used by the remote expert RE may not provide sufficient information for the remote expert RE to fully assess the status of the imaging examination.

To address such problems, as disclosed herein, an image processing module 32 is provided for processing images acquired by the medical imaging device 2 as a portion of a method or process 100 of providing assistance to the local operator during a medical imaging examination. The images are transferred from the medical imaging device controller 10 (operable by the local operator LO) to the remote workstation 12 (operable by the remote expert RE) via the communication link 14. In one embodiment, the acquired images are processed by the at least one electronic processor 20′ of the medical imaging device controller 10 before transmission to the remote workstation 12. That is, the image processing module 32 is implemented in the medical imaging device controller 10. In another embodiment, the acquired images are processed by the at least one electronic processor 20 of the remote workstation 12 after transmission from the medical imaging device controller 10. That is, the image processing module 32 is implemented in the remote workstation 12. For brevity, the assistance method 100 is described herein terms of the image processing module 32 being implemented in the remote workstation 12, as shown in FIG. 1.

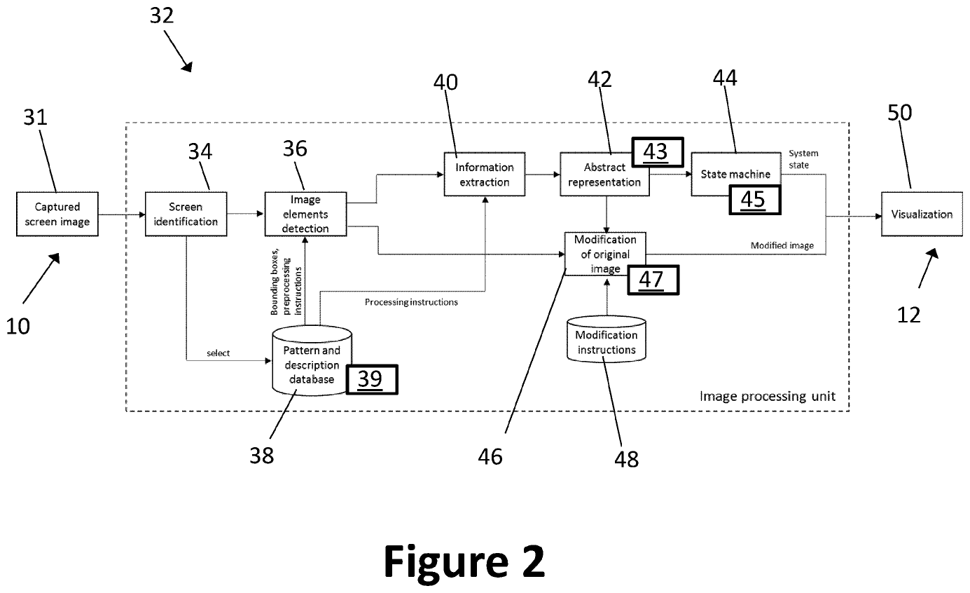

Referring now to FIG. 2, and with continuing reference to FIG. 1, an example of the image processing module 32 is shown. A captured screen image 31 (e.g., a video frame from the video feed 17 or the screen mirroring data stream 18) is input to the image processing module 32. A screen identification module 34 is configured to identify a screen or view of the captured screen image 31. Many screens images 31 on the GUI 28′ of the medical imaging device controller 10 offer different screens or views, or windows and dialogs can be shown on top of the GUI. For example, on an MR console (i.e., the medical imaging device 2), the local technician LO could select one of a plurality of screens 31 that display different information. While one screen shows the patient information, another screen may show the details of the medical imaging examination. The screen identification module 34 is configured to detect the particular screen presented in the captured screen image 31 by, for example, picking a specific region of the captured screen image 31 that serves as a unique identifier of the captured screen image. For example, the specific region of the captured screen image 31 can be, for example, a color, or a specific element in the image. In some examples, the screen identification module 34 can be comprises a machine-learning module configured to identify screens, with multiple instances of the screens displaying different information being used as training data. The vendor of the medical imaging device 2, the modality of the medical imaging device, and/or a version of the UI in some embodiments is also detected by the screen identification module 34. However, these pieces of information are already available in some cases (for example, provided by a workflow scheduler of the ROCC which initiated the connection between the local operator and the remote expert), so that in these cases the screen identification module 34 only needs to distinguish between a relatively small number of different screens provided by the (in these cases a priori-known) make and model of the imaging device 2.

An image element detection module 36 is configured to identify the screen regions of the identified screen containing desired information. To do so, the image element detection module 36 retrieves one or more templates 39 of the information from the screens from a pattern and description database 38. The templates 39 includes information related to the content of the screens along with a position of information on the screen. The image element detection module 36 uses the identified screens from the screen identification module 34 to pre-select the templates 39 from the pattern and description database 38 that belong to the identified screens. The types of templates 39 stored in the pattern and description database 38 can include for each type of displayed user interface (e.g., vendor and software version of the medical imaging device 2) multiple items of information, including, for example, possible positions of information on the captured screens 31; labels of information (e.g., remaining exam time, number of scans, type of radiofrequency (RF) coil used, and so forth); type of information (e.g., to be extracted, to be deleted/modified, to be highlighted, and so forth); type of encoding of information (e.g. text, number, icon, progress bar, color, and so forth); for text or numbers, formatting of this information (e.g., time displayed using in seconds or minutes, using decimals, etc.) and text style (font type and size, text alignment and line breaks, etc.); for icons or symbols, a translation table icon/pattern to meaning; for a progress bar, a shape and color of progress bar and surrounding box; for color, a translation table color to meaning, and so forth. These are merely examples, and should not be construed as limiting. The templates 39 of the pattern and description database 38 can be updated every time a new user interface is included.

An information extraction module 40 is configured to extract the image elements detected by the image elements detection module 36 from respective patches of image data. To do so, in one example, the information extraction module 40 can perform an optical character recognition (OCR) process can be used to identify text or numbers. For colors, the information extraction module 40 can extract mean, red, green, and blue values of an image patch of the captured screen image 31. For icons or symbols, the information extraction module 40 can perform a pattern comparison with images stored in the pattern and description database 38. The pattern and description database 38 further includes information about how to interpret the extracted information, e.g. by providing translation tables from colors/icons to meaning. The information extraction module 40 is configured to convert the extracted pieces of information to a correct form and labelled according to the information in the pattern and description database 38.

The use of image elements detection 36 followed by extraction of information from the image elements 40 is one approach. However, other approaches can be used to extract the information, such as omitting the regions identification (i.e., the image elements detection module 36) and employing OCR and/or image matching applied to the captured screen image 31 as a whole.

The image processing module 32 operates in (near) real time to extract information from successive captured screen images 31 (e.g., from successive video frames of the video feed 17 or the screen mirroring data stream 18). This may involve analyzing every video frame of the video feed, or a subset of the video frames. For example, if the video has a frame rate of 30 frames/sec (30 fps), it may be sufficient to process every sixth frame thereby providing a temporal resolution of ⅕^{th} of a second while greatly reducing total amount of processing. By such processing of successive image frames, the image processing module 32 extracts information from various screens of the GUI 28′ of the medical imaging device controller 10, as the local operator LO navigates amongst these various screens. For example, in a typical workflow, the local operator LO may initially bring up one or more imaging examination setup screens via which the imaged anatomy and specific imaging sequences/scans are selected/entered; thereafter, the local operator may move to the scan/sequence setup screen(s) to set parameters of the imaging scan or sequence; thereafter the local operator may move to the scout scan screen to acquire a scout scan for determining the imaging volume; thereafter the local operator may move to the image acquisition screen; and so forth. As the user navigates through these various screens and enters relevant data, the image processing module 32 successively applies the operations 34, 36, 40 to extract the information from each successively navigated screen. From this collection of extracted information, an abstract generation module 42 is configured to create a representation 43 of the extracted features by inserting the converted pieces of information into a generic data structure that is identical for all types of imaging modalities, systems, and user interfaces. The data structure contains elements such as number of scans, remaining scan time, patient weight, time from start of exam, number of rescans, name of scan protocol, progress of running examination, heart rate, breathing rate, etc. If a required piece of information is not available on a user interface, the corresponding element of the data structure is left empty, marked “not available”, or filled with a default value.

In one embodiment, the abstract representation 43 serves as a persistent representation of the current state of the imaging examination. Alternatively, further processing may be performed. In the illustrative example of FIG. 2, in this further processing, the abstract representation 43 of status information is used as an input to a state machine module 44 to generate an imaging examination workflow model 45 of a status (i.e. state) of the medical imaging examination (more generally, the workflow model 45 can be any other suitable model, such as a Business Process Model Notation (BPMN) model). The state machine module 44 stores the current status and parameters of the medical imaging device 2 and the medical imaging device controller 10, even when not all information is visible on the display device 24′ at all times. For example, the state machine module 44 may receive the information that a new patient case has been created in one screen of the user interface displayed on the medical imaging device controller 10. After that, the local operator LO changes the screen on the medical imaging device controller 10 to enter the protocol information. The state machine module 44 stores the patient information and the point in time when the medical imaging examination was initiated. The state machine module 44 later receives information about the progress of the data acquisition and the remaining scan time. Even when the local operator LO views a different window on top or switched between user interface screens, the progress information and exam status are still stored in the state machine module 44. The state machine module 44 uses this data to generate the imaging examination workflow model 45.

Concurrently, or at different times, in some embodiments after the captured screen image 31 is processed by the image elements detection module 36, the detected image elements are also used by an image modification module 46 to generate one or more modified images 47 from the captured screen image 31. The image elements are deleted from the captured screen image 31, modified in the captured screen image 31, highlighted or otherwise annotated in the captured screen image 31 by the image modification module 46 in order to create the modified image 47. Deletions can be used to remove patient-identifying information (PII) or other information that is preferably not shown to the remote expert RE. Highlighting or other annotation can be used to draw attention to selected items shown in the screen. In one approach, the screen regions identified by the templates 39 are marked as to how the modifications are to be done. For example, the image modification module 46 is configured to: (i) either remove image elements from the captured screen image 31 (if marked “to be deleted”); (ii) replaces image elements by other information (if marked “to be modified”), or (iii) highlight the information on the captured screen image (if marked “to be highlighted”). In the example of modification, instructions on how this modification is to be done and what the element should be replaced with is read from a modification instructions database 48 (which may be associated with the templates 39). Some examples for modification instruction can include “replace element labelled “patient name” by text “ANONYMOUS”. In addition to fixed text or symbols, replacement elements can also be derived from the abstract representation 43. In the case of highlighting, the corresponding part of the captured screen image 31 is either marked by a frame or highlight color, or the rest of the captured screen image is darkened or distorted. Highlighting can be used for training purposes or for guiding the operator to the next action or currently important information. These operations are used to generate the modified images 47.

A visualization 50 is generated by the image processing module 32 for display on the display device 24 of the remote workstation 12. The visualization includes one or more of the representation 43 generated by the abstract representation module 42, the representation of the state machine 45 generated by the state machine module 44, and the modified images 47 generated by the image modification module 46, or any overlay of any of these options. The remote expert RE can select how the visualization 50 is displayed on the workstation 12. The representation of the state machine module 44 can be used to create different kinds of visualizations. In addition, since the data structure used to generate the abstract representation 42 is the same for all the different user interfaces of the local medical imaging devices 2, the information can be displayed in a generic way that allows the remote expert RE to quickly understand the status of the medical imaging examination.

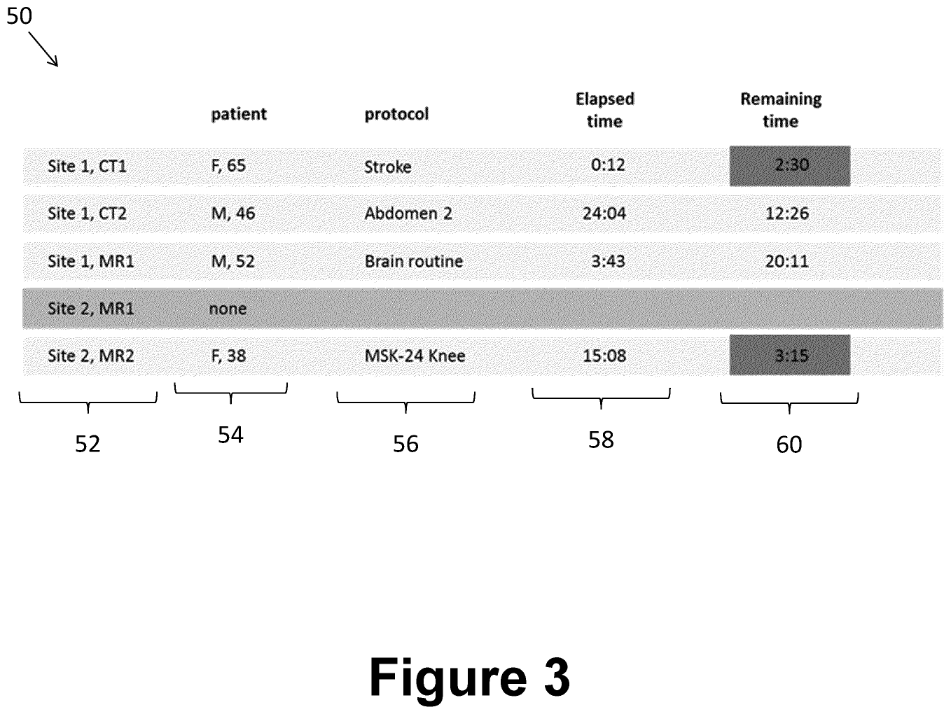

In some examples, status information from medical imaging device controllers 10 can be displayed simultaneously in a structured form in the visualization 50 at the remote workstation 12, for example as a table or as multiple rows or columns of display elements. FIG. 3 shows an example of the visualization 50. As shown in FIG. 3, the visualization 50 shows five fields: a location field 52 showing a location, modality, and identification of the medical imaging device 2, a patient field 54 showing a gender and age of the patient undergoing the medical imaging examination, a protocol field 56 showing a type of medical imaging examination, an elapsed time field 58 showing the elapsed time of the medical imaging examination, and a remaining time field 60 showing the time remaining for the medical imaging examination. In some examples, the remaining time field 60 entries can be annotated (e.g., highlighted) when the remaining time approaches zero, in which case the medical imaging examination is completed.

Referring back to FIG. 2, in other examples, the abstract representation 42 can be used for triggering automated actions and/or processes. For example, the extracted information can be used for automatically alerting the remote expert RE (or any other person involved in the process) about a next action to be taken, about a possible schedule conflict, about an expected delay for the next action, about a change in the order of actions, about the time to the next action, etc. In another example, the abstract representation 42 can be further forwarded to an automated prediction or adaptive scheduling engine (not shown). For example, the remaining scan times extracted from a number of different medical imaging devices 2 can be used to automatically rearrange a schedule and create task prioritizations for a radiology department or for the remote expert RE. In a further example, the abstract representation 42 can be used to detect deviations from standard procedures and either document the deviation for quality assurance reasons or alert the remote expert RE about the deviation. For example, deviations from protocols of the medical imaging examination happen when the local operator LO removes or adds an imaging sequence for the ongoing examination or changes any of the image contrast settings.

The non-transitory computer readable medium 26 of the remote workstation 12 can store instructions executable by at least one electronic processor 20 to perform the method 100 of providing assistance from the remote expert RE to a local operator LO of a medical imaging device 2 during the medical imaging examination. Stated another way, the non-transitory computer readable medium 26 of the remote workstation 12 stores instructions related to the implementation of the image processing module 32.

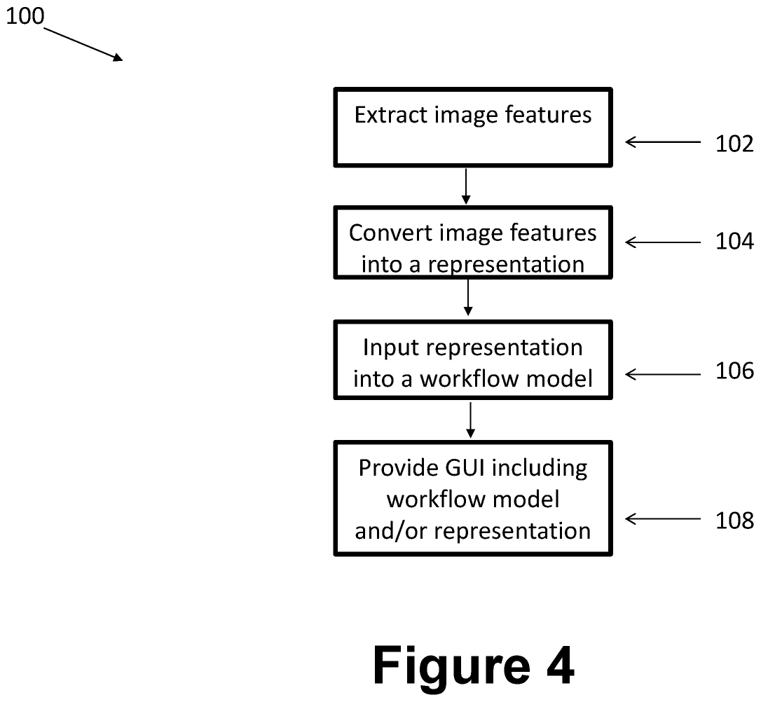

With reference to FIG. 4, and with continuing reference to FIGS. 1-3, an illustrative embodiment of the assist method 100 is diagrammatically shown as a flowchart. To begin the assist method 100, one or more images of a patient are acquired by the medical imaging device 2 operated by the local operator LO during a medical imaging examination. The images and/or settings related to the medical imaging examination are shown on the display device 24′ of the medical imaging device controller 10. At an operation 102, image features from image frames displayed on the medical imaging device controller 10 are extracted. The operation 102 can be performed by the screen identification module 34, the image elements detection module 36 (in conjunction with the pattern and description database 38), and the information extraction module 40.

In one example, the image features can be extracted using the screen sharing device 13 (i.e., running screensharing software) of the medical imaging device controller 10 with the remote workstation 12. In another example, the video feed 17 of the medical imaging device controller 10 is captured by the camera 16 and transmitted to the remote workstation 12. The image features are extracted by the remote workstation 12 from the received video feed 17. The extracted information from the image features includes one or more of: position of image features on the display device 24′ of the medical imaging device controller 10; textual labels of the image features; type of information of the image features; type of encoding of the image features; type of formatting of the image features; a translation table or icon of the image features; and a shape or color of the image features, and so forth.

The extracting operation 102 can be performed in a variety of manners. In one example, the extraction includes performing an OCR process on the image frames to extract textual information. In another example, mean color values of the image frames are extracted to extract color information. In a further example, a pattern comparison operation is performed on the image with images stored in a database (e.g., the pattern and description database 38) to extract the image features. In yet another example, a corresponding dialog screen template 39 that corresponds to a dialog screen depicted in an image frame is identified. The corresponding dialog screen template 39 identifies one or more screen regions and associates the one or more screen regions with settings of the medical imaging examination. The extracted image features are extracted from the image frames and associated extracted information in the one or more screen regions with settings of the medical imaging examination using the associations provided by the corresponding dialog screen template 39.

At an operation 104, the extracted image features are converted into a representation 43 (i.e., the abstract representation) of a current status of the medical imaging examination. The operation 104 is performed by the abstract representation module 42. To generate the representation 43, the extracted image features are input into a generic imaging examination workflow model that is independent of a format of the image features displayed on the display device 24′ of the medical imaging device controller 10. The representation 43 includes one or more of: a number of scans, a remaining scan time, a weight value of a patient to be scanned, a time elapsed since a start of the medical imaging examination, a number of rescans, a name of a scan protocol, a progress of a current medical imaging examination, a heart rate of the patient to be scanned, and a breathing rate of the patient to be scanned.

In some examples, the operation 104 can include operations performed by the image modification module 46. To do so, one of more of the extracted features from the image frames are identified as personally identifiable information of the patient to be scanned during the medical imaging examination. One or more modified image frames comprising the modified images 47 displayed on the display device 24′ of the medical imaging device controller 10 are generated by one of removing the identified personally identifiable information features from the image frames or replacing the personally identifiable information in the image frames with text, a symbol, or a color. The modified image frames 47 are displayed as a video feed on the GUI 28 on the workstation 12.

At an operation 106, the representation 43 is input into an imaging examination workflow model 45 indicative of a current state of the medical imaging examination. The operation 106 is performed by the state machine module 44. The imaging examination workflow model 45 is then provided on the remote workstation 12. In some examples, the extracted image features include data input to the medical imaging device controller 10 and displayed on the display device 24′. The imaging examination workflow model 45 is then updated with this inputted data. In another example, a trigger event in the imaging examination workflow model 45 can be identified, at which an action needs to be taken by the remote expert RE and/or the local operator LO. An alert 30 indicating the trigger event can then be output via the GUI 28 of the remote workstation 12.

At an operation 108, the GUI 28 is configured to display the visualization 50 (e.g., one or more of the representation 43 generated by the abstract representation module 42, the representation of the state machine 45 generated by the state machine module 44, and the modified images 47 generated by the image modification module 46, or any overlay of any of these options). The visualization 50 can be displayed using a standard display format that is independent of the medical imaging device 2 operated by the local operator LO during the medical imaging examination.

Although primarily described in terms of a single medical imaging device bay 3 housing a single medical imaging device 2, the method 100 can be performed at a plurality of sites including medical imaging devices operated by a corresponding number of local operators, and the visualization 50 can include information from the sites of the plurality of sites. The visualization 50 includes a list displayed at the remote workstation 12 showing a status of the medical imaging examinations at the corresponding sites, such as the one shown in FIG. 3. This is of particular benefit to a remote expert RE who is concurrently monitoring and/or assisting multiple imaging bays, possibly having imaging devices of different makes and/or models. The representation 43 provides the remote expert RE with a device-independent summary of pertinent information about the state of the imaging examination being conducted in each imaging bay, while the modified image frames 47 (shown in time sequence as the image processing module 32 process successive captured screen images 31) provides (modified) mirrored video of the imaging device controller. In a typical implementation, the representation 43 may be shown at all times to provide status information on all monitored imaging bays; while, the video comprising the modified image frames 47 are shown for one particular imaging bay to which the remote expert RE is currently providing assistance. In this way, the remote expert RE has detailed current situational awareness of the bay being assisted, while the remote expert RE also maintains awareness of the statuses of all imaging bays assigned to that remote expert.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of providing assistance from a remote expert (RE) to a local operator (LO) of a medical imaging device during a medical imaging examination, the method comprising:
extracting image features from image frames displayed on a display device of a controller of the medical imaging device operable by the local operator during the medical imaging examination;
converting the extracted image features into a representation of a current status of the medical imaging examination; and
providing a user interface (UI) displaying the representation on a workstation operable by the remote expert.

2. The non-transitory computer readable medium of claim 1, wherein converting the extracted image features into the representation of the current status of the medical imaging examination further includes:
inputting the extracted image features into a generic imaging examination workflow model that is independent of a format of the image features displayed on the display device of the controller operable by the local operator (LO) to generate the representation.

3. The non-transitory computer readable medium of claim 1, wherein the representation includes one or more of: a number of scans, a remaining scan time, a weight value of a patient to be scanned, a time elapsed since a start of the medical imaging examination, a number of rescans, a name of a scan protocol, a progress of a current medical imaging examination, a heart rate of the patient to be scanned, and a breathing rate of the patient to be scanned.

4. The non-transitory computer readable medium of claim 1, wherein the method further includes:
inputting the representation into an imaging examination workflow model indicative of a current state of the medical imaging examination; and
providing the imaging examination workflow model on the workstation operable by the remote expert (RE).

5. The non-transitory computer readable medium of claim 4, wherein the extracted image features include data input to the controller by the local operator (LO) and displayed on the display device of the controller, and the method further includes:
updating the imaging examination workflow model provided on the workstation operable by the remote expert (RE) with the data input by the local operator.

6. The non-transitory computer readable medium of claim 4, wherein the method further includes:
identifying a trigger event in the imaging examination workflow model at which an action needs to be taken by the remote expert (RE) and/or the local operator (LO); and
outputting an alert via the UI operable by the remote expert indicating the trigger event.

7. The non-transitory computer readable medium of claim 1, wherein converting the extracted image features into a representation of a current status of the medical imaging examination further includes:
identifying one of more of the extracted features from the image frames as personally identifiable information of a patient to be scanned during the medical imaging examination;
generating modified image frames from the image frames displayed on the display device of the controller by one of removing the identified personally identifiable information features from the image frames or replacing the personally identifiable information in the image frames with text, a symbol, or a color; and
displaying the modified image frames as a video feed presented on the UI on the workstation operated by the remote expert (RE).

8. The non-transitory computer readable medium of claim 1, wherein extracting image features from image frames displayed on the display device of the controller operable by the local operator (LO) during the medical imaging examination further includes:
identifying a corresponding dialog screen template that corresponds to a dialog screen depicted in an image frame wherein the corresponding dialog screen template identifies one or more screen regions and associates the one or more screen regions with settings of the medical imaging examination; and
extracting information from the image frame and associating the extracted information in the one or more screen regions with settings of the medical imaging examination using the associations provided by the corresponding dialog screen template.

9. The non-transitory computer readable medium of claim 1, wherein the extracted information includes one or more of: position of image features on the display device; textual labels of the image features; type of information of the image features; type of encoding of the image features; type of formatting of the image features; a translation table or icon of the image features; and a shape or color of the image features.

10. The non-transitory computer readable medium of claim 1, wherein extracting image features from image frames displayed on the display device of the controller operable by the local operator (LO) during the medical imaging examination includes at least one of:
performing optical character recognition (OCR) on the image frames displayed on the display device of the controller operable by the local operator to extract textual information;
extracting mean color values on the image frames displayed on the display device of the controller operable by the local operator to extract color information; and
performing a pattern comparison operation on the image frames displayed on the display device of the controller operable by the local operator with images stored in a database.

11. The non-transitory computer readable medium according to claim 1, wherein the method further includes:
extracting the image features from the image frames displayed on the display device of the controller using screen sharing software running on the controller.

12. The non-transitory computer readable medium according to claim 1, wherein the method further includes:
at the workstation operated by the remote expert (RE), receiving a video feed capturing the display device of the controller operated by the local operator (LO);
displaying the video feed at the workstation operated by the remote expert; and
extracting the image features from the received video feed.

13. The non-transitory computer readable medium according to claim 1, wherein the method further includes:
displaying the representation at the workstation operated by the remote expert (RE) using a standard display format that is independent of the medical imaging device operated by the local operator (LO) during the medical imaging examination.

14. The non-transitory computer readable medium according to claim 1, wherein the method is performed at a plurality of sites including medical imaging devices operated by a corresponding number of local operators (LO), and the representation include information from the sites of the plurality of sites.

15. The non-transitory computer readable medium of claim 14, wherein the representation includes a list showing a status of the medical imaging examinations at the corresponding sites.

16. An apparatus for providing assistance from a remote expert (RE) to a local operator (LO) during a medical imaging examination performed using a medical imaging device, the apparatus comprising:
a workstation operable by the remote expert; and
at least one electronic processor programmed to:
extract image features from image frames displayed on a display device of a controller of the medical imaging device operable by the local operator during the medical imaging examination;
convert the extracted image features into a representation of a current status of the medical imaging examination by inputting the image features into an imaging examination workflow model indicative of a current state of the medical imaging examination; and
provide a user interface (UI) displaying at least one of the representation and the imaging examination workflow model on the workstation operable by the remote expert.

17. The apparatus of claim 16, wherein the at least one electronic processor is further programmed to:
input the extracted image features into a imaging examination workflow model having a standard format that is independent of a format of the image features displayed on the display device operable by the local operator (LO) to generate the representation.

18. The apparatus of claim 16, wherein the at least one electronic processor is further programmed to:
output an alert when the imaging examination workflow model reaches a procedure in the medical imaging examination where an action needs to be taken by either the remote expert (RE) or the local operator (LO).

19. The apparatus of claim 16, wherein the at least one electronic processor is further programmed to convert the extracted image features into a representation of a current status of the medical imaging examination by:
identifying one of more of the extracted features from the image frames as personally identifiable information of a patient to be scanned during the medical imaging examination;
generating modified image frames from the image frames displayed on the display device of the controller by one of removing the identified personally identifiable information features from the image frames or replacing the personally identifiable information in the image frames with text, a symbol, or a color; and
displaying the modified image frames as a video feed presented on the UI on the workstation operated by the remote expert (RE).

20. A method of providing assistance from a remote expert (RE) to a local operator (LO) during a medical imaging examination, the method comprising:
extracting image features from image frames displayed on a display device of a controller operable by the local operator during the medical imaging examination;
converting the extracted image features into a representation indicative of a current status of the medical imaging examination by:
identifying one of more of the extracted features from the image frames as personally identifiable information of a patient to be scanned during the medical imaging examination; and
generating modified image frames from the image frames displayed on the display device of the controller by one of removing the identified personally identifiable information features from the image frames or replacing the personally identifiable information in the image frames with text, a symbol, or a color;
inputting the representation into an imaging examination workflow model indicative of a current state of the medical imaging examination; and
providing a user interface (UI) displaying the modified image frames as a video feed, the abstract representation, and the imaging examination workflow model on a workstation operable by the remote expert.
